(19) United States
(12) Patent Application Publication
(10) Pub. No.: US 2009/0156986 A1
(43) Pub. Date: Jun. 18, 2009

(54) ROTATOR CUFF PATCH DELIVERY DEVICE

(75) Inventor: SCOTT TRENHAILE, Belvidere, IL (US)

Correspondence Address: DICKE, BILLIG & CZAJA, FIFTH STREET TOWERS, 100 SOUTH FIFTH STREET, SUITE 2250, MINNEAPOLIS, MN, 55402, US

(73) Assignee: ROCKFORD ORTHOPAEDIC SPORTS MEDICINE SERVICES, LLC, Belvidere, IL (US)

(21) Appl. No.: 12/333,880

(22) Filed: Dec. 12, 2008

Related U.S. Application Data
(60) Provisional application No. 61/012,999, filed on Dec. 12, 2007.

(51) Int. Cl. A61M 31/00

(52) U.S. Cl. 604/48; 604/59; 604/93.01

(57) ABSTRACT

A device for delivering an allograft patch through a cannula provides for feeding of the device and the allograft patch through the cannula and provides for deploying of the allograft patch after feeding through the cannula.

### CROSS-REFERENCE TO RELATED APPLICATIONS

This application claims priority under 35 U.S.C. §119(e) to U.S. Provisional Patent Application Ser. No. 61/012,999, filed on Dec. 12, 2007, and incorporated herein by reference. This application is related to U.S. Non-Provisional patent application Ser. No. XX/XXX,XXX, filed on even date herewith, having attorney docket number R615.102.102, and incorporated herein by reference.

### FIELD OF THE INVENTION

The present invention relates generally to a medical device that facilitates orthopaedic surgeons of varying technical skill to perform an “all-arthroscopic” allograft patch augmentation after a rotator cuff repair. The device would address the technical challenge of introducing the allograft patch through a cannula and then positioning the graft with appropriate tension and coverage once it is placed in the subacromial space.

### BACKGROUND OF THE INVENTION

Rotator cuff tears that are massive, unduly stiff, and/or chronic in nature are difficult to repair surgically. Historically these tears have been left alone, debrided, or surgically addressed in an open manner. More recently, some surgeons have attempted to increase healing rates and success of such rotator cuff repairs by augmenting the repair with allograft tissue. A patch or graft of allograft tissue is placed over the top of the native rotator cuff tendon and bone to reinforce the repair for load bearing and thickening of the tendon.

This augmentation has routinely been done through a formal open approach requiring partial detachment of the deltoid for visualization. Of the shoulder musculature that remains after a rotator cuff tear, the deltoid is usually one of the only remaining muscles that still functions well. Detaching this tendonous origin from the acromion can raise a myriad of complications and morbidity including secondary detachment, axillary nerve injury, and anterior superior escape.

Performing an all-arthroscopic rotator cuff repair along with allograft augmentation reduces the complications of the open approach and decreases morbidity. Furthermore, visualization is improved by using the arthroscope to magnify the native rotator cuff tissue and facilitate repair and augmentation placement. One primary challenge with this type of arthroscopic approach, however, is delivering the allograft patch into the subacromial space and getting the allograft patch spread out over the native rotator cuff tissue with the appropriate tension and coverage. In addition, arthroscopic fluid can produce turbulence around the patch creating entanglement and difficult visualization.

For these and other reasons, there is a need for the present invention.

### BRIEF DESCRIPTION OF THE DRAWINGS

FIG. 1 illustrates one existing technique of performing a rotator cuff repair.

FIG. 2 illustrates one embodiment of a rotator cuff patch delivery device according to the present invention.

FIG. 3 illustrates one embodiment of delivery of a rotator cuff patch with the rotator cuff patch delivery device of FIG. 2.

FIG. 4 illustrates one technique of performing a rotator cuff repair using the rotator cuff patch delivery device of FIG. 2.

FIG. 5 illustrates another embodiment of a rotator cuff patch delivery device according to the present invention.

FIG. 6 illustrates one technique of performing a rotator cuff repair using the rotator cuff patch delivery device of FIG. 5.

FIG. 7 illustrates another embodiment of the rotator cuff patch delivery device of FIG. 5.

FIGS. 8 and 9 illustrate another embodiment of a rotator cuff patch delivery device according to the present invention.

FIGS. 10 and 11 illustrate one embodiment of a rotator cuff patch secured to the rotator cuff patch delivery device of FIGS. 8 and 9.

FIG. 12 illustrates one embodiment of a rotator cuff patch secured to the rotator cuff patch delivery device of FIGS. 8 and 9 in a retracted state.

FIG. 13 illustrates one embodiment of delivery of a rotator cuff patch with the rotator cuff patch delivery device of FIGS. 8 and 9.

FIG. 14 illustrates one embodiment of a rotator cuff patch secured to the rotator cuff patch delivery device of FIGS. 8 and 9 in a deployed state.

FIGS. 15, 16, and 17 illustrate another embodiment of a rotator cuff patch delivery device according to the present invention.

FIG. 18 illustrates one embodiment of the rotator cuff patch delivery device of FIGS. 15, 16, and 17 in a retracted state.

FIGS. 19 and 20 illustrate one embodiment of a rotator cuff patch secured to the rotator cuff patch delivery device of FIGS. 15, 16, and 17.

FIG. 21 illustrates one embodiment of a rotator cuff patch secured to the rotator cuff patch delivery device of FIGS. 15, 16, and 17 in a retracted state.

FIG. 22 illustrates one embodiment of delivery of a rotator cuff patch with the rotator cuff patch delivery device of FIGS. 15, 16, and 17.

FIG. 23 illustrates one embodiment of a rotator cuff patch secured to the rotator cuff patch delivery device of FIGS. 15, 16, and 17 in a deployed state.

### DETAILED DESCRIPTION

A desirable device to perform an “all-arthroscopic” allograft patch augmentation after a rotator cuff repair would allow the surgeon to deliver the allograft patch through a standard arthroscopic cannula. Once in the subacromial space, the device would then assist in positioning of the patch in a controllable fashion and placement of the graft.

### Existing Techniques

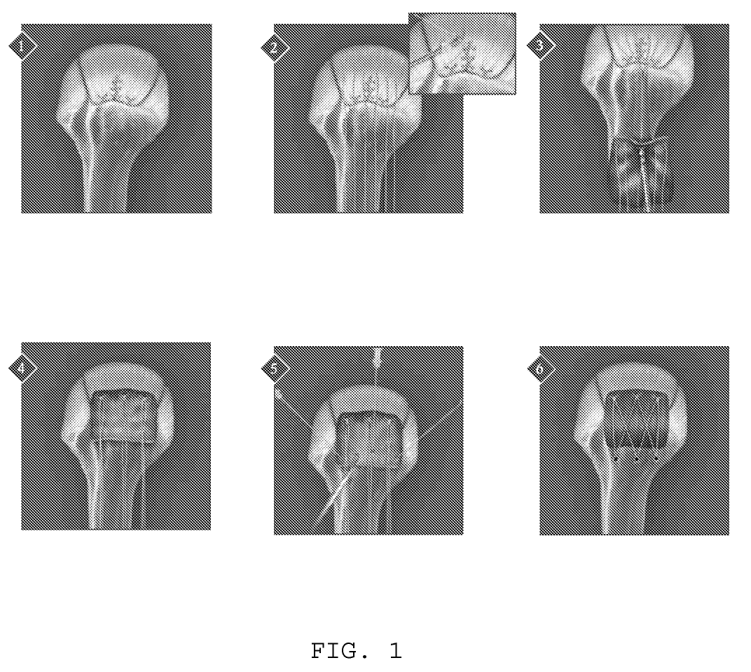

With reference to FIG. 1, one existing technique of performing a rotator cuff repair is described. To start the rotator cuff repair, the patient may be positioned in the lateral decubitus or beach chair position. In one approach, routine anterior, lateral, and posterior shoulder portals are established. Intra-articular pathology is addressed prior to entering the subacromial space. The rotator cuff tear is then mobilized and repaired arthroscopically (FIG. 1, image 1).

In one approach, the area to be covered by the allograft patch is measured with a graduated probe through the lateral and posterior portals to gain medial to lateral and anterior to posterior measurements respectively. In one approach, three non-absorbable sutures are placed in the medial most portion of the rotator cuff tendon near the myotendonous junction within the area to be covered by the allograft patch (FIG. 1, image 2). In one approach, the sutures are placed in a horizontal fashion, and the six free suture tails or limbs are then passed outside the body through a lateral portal through a cannula. In one approach, using a free needle, the six suture limbs are then passed through the medial edge of the patch in a horizontal mattress fashion outside of the shoulder. In one approach, the midline suture is then tied using an arthroscopic knot outside of the body.

In one approach of one existing technique, a knot pusher is then utilized to push the allograft patch through the cannula with the pre-tied arthroscopic knot (FIG. 1, image 3). Once the patch and knot reach the subacromial space, the knot can be tightened into position. The anterior and posterior suture limbs can then be retrieved through the anterior and posterior respective portals and tied respectively (FIG. 1, image 4). In one approach, using spinal needles, the lateral portion of the patch can be provisionally fixed to the tuberosity of the humerus bone (FIG. 1, image 5). In one approach, the medial suture limbs are then brought over the top of the patch in a criss-cross fashion and secured using a “double-row” fixation anchor. The spinal needles may then be removed and the repair is complete (FIG. 1, image 6).

A difficulty with the procedure described above is the point at which the allograft patch is delivered into the subacromial space and has to be “rolled out” into position. Because of the constant flow of arthroscopic fluid that is present, turbulence and compression of the graft through the cannula can cause entanglement of the patch. One method to assist in unrolling the graft and regaining orientation is to place a series of stripes on the superior side of the patch with a marker. This gives the surgeon visual feedback as to whether the graft is flipped or rolled in any way prior to placing the sutures from the medial row over the top to the lateral row. While this is helpful, it is not enough for the novice surgeon. An assistive device would be beneficial to help in delivering and laying out and/or positioning of the graft in a flat position once in the subacromial space.

### Rotator Cuff Patch Delivery Devices

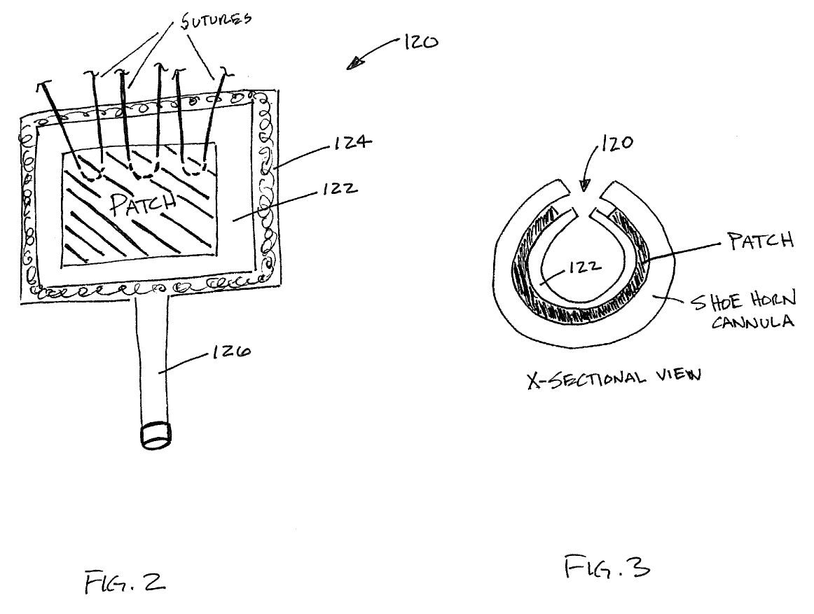

FIG. 2 illustrates one embodiment of a rotator cuff patch delivery device according to the present invention. The rotator cuff patch delivery device of FIG. 2 includes a central pad portion and an inflatable chamber or bladder portion provided around a perimeter of the pad portion. In one embodiment, a fluid influx tube extends from the inflatable chamber portion. The fluid influx tube communicates with the inflatable chamber portion and has an open end to provide for selective inflation of the inflatable chamber portion, as described below.

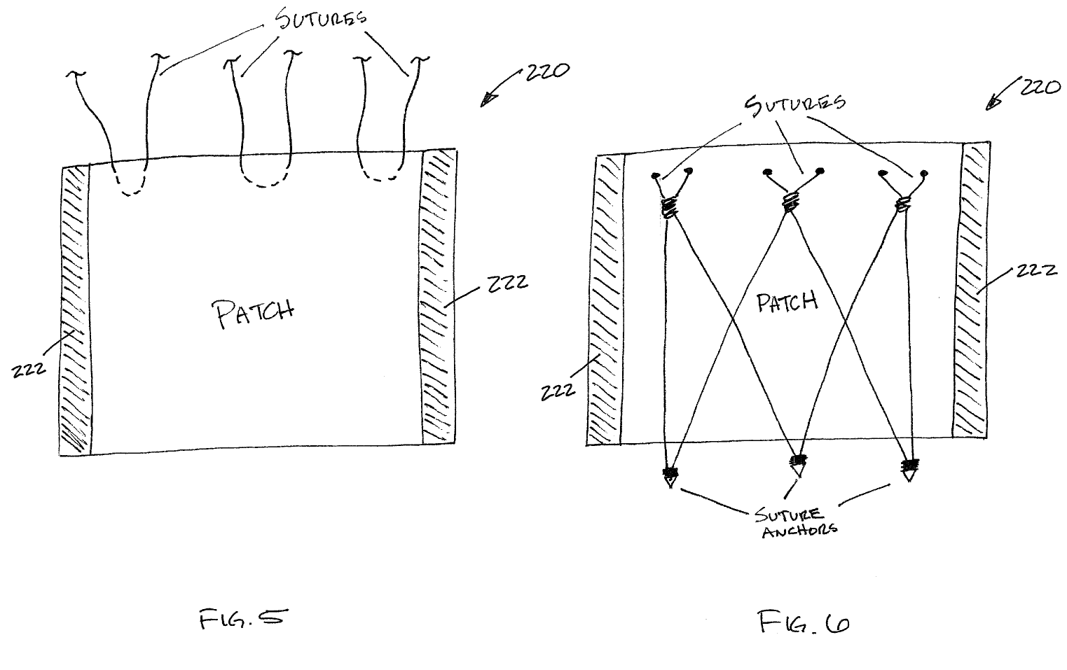

In one embodiment of use, with the inflatable chamber portion of the rotator cuff patch delivery device deflated, the patch is secured to the pad portion with sutures such that a surface of the pad portion is adjacent the superior surface of the patch (FIGS. 2 and 4). In one embodiment, the pad portion and the patch adhere to each other when wet.

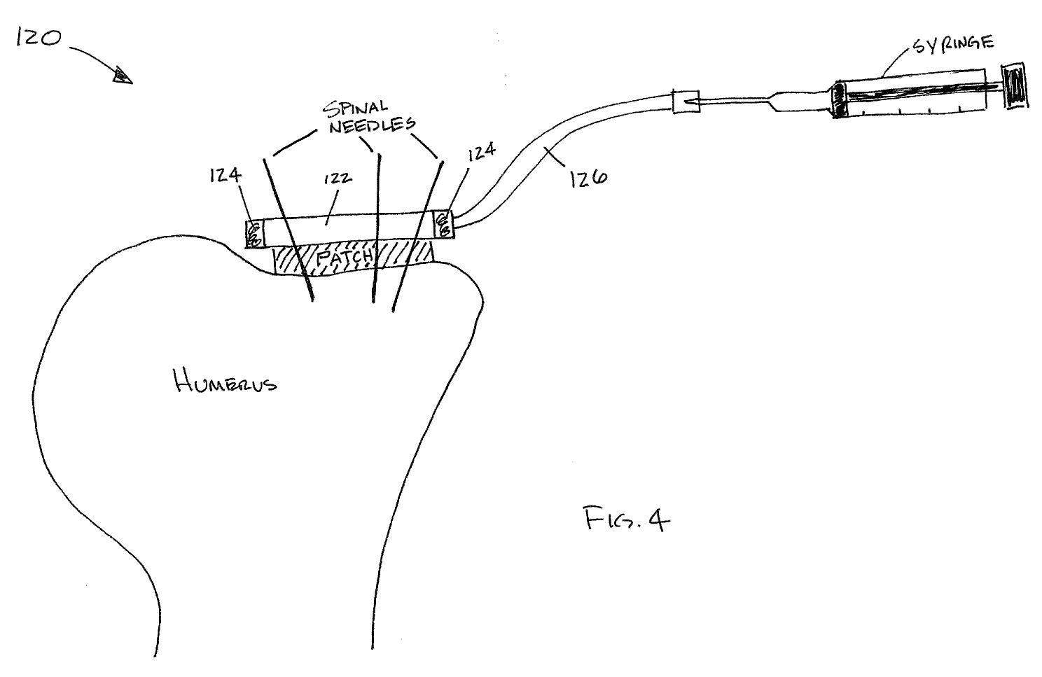

In one embodiment, with the inflatable chamber portion of the rotator cuff patch delivery device deflated and the patch secured to the pad portion, free suture tails or limbs from sutures placed in the medial most portion of the rotator cuff tendon, as described above, are passed outside the body. Thereafter, the whole construct, including the patch and the rotator cuff patch delivery device, is rolled up longitudinally and fed into the cannula (FIG. 3). The whole construct, including the patch and the rotator cuff patch delivery device, is then delivered into the subacromial space of the joint (FIG. 4).

Once inside the joint, the inflatable chamber portion of the rotator cuff patch delivery device is inflated to deploy the patch. In one embodiment, the inflatable chamber portion is inflated via the fluid influx tube. In one embodiment, for example, a syringe outside the body is connected with the open end of the fluid influx tube and air (or liquid) is introduced into the inflatable chamber portion with the syringe. Accordingly, the air (or liquid) inflates the inflatable chamber portion and “unrolls” the pad portion, thereby deploying the patch such that the patch returns to its original rectangular shape. As such, the inflated rotator cuff patch delivery device adds rigidity to the patch, and controls twisting and flipping of the patch during positioning within the joint.

In one embodiment, the medial row of sutures exiting the patch pierce the pad portion in an area spaced from the inflatable chamber portion. These sutures are then tied down to complete medial fixation. In one embodiment, spinal needles are inserted through the central pad portion (i.e., non-air chamber portion) of the rotator cuff patch delivery device to provide provisional fixation. A lateral row of anchors can then be used to anchor the sutures in a standard fashion.

In one embodiment, the fluid influx tube is cut off at the communication with the inflatable chamber portion and removed. In one embodiment, the rotator cuff patch delivery device is formed of a bioabsorbable material with a short half-life so as to degrade over time. An example of such a material includes corn starch.

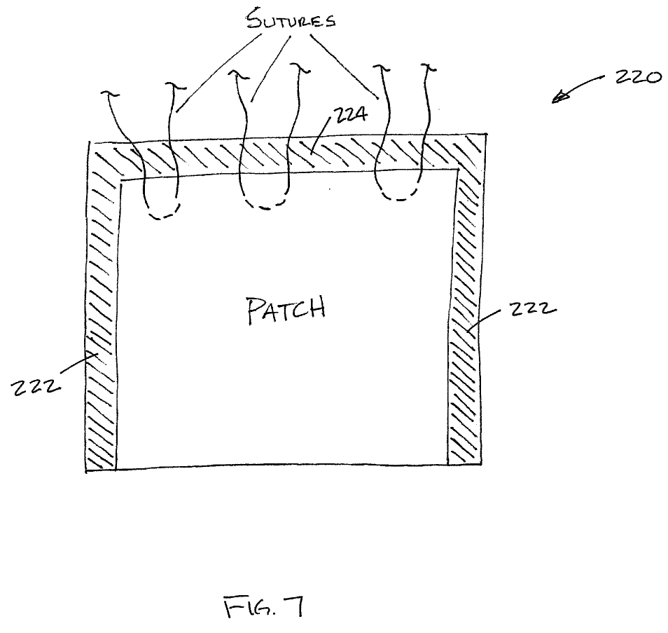

FIG. 5 illustrates another embodiment of a rotator cuff patch delivery device according to the present invention. The rotator cuff patch delivery device of FIG. 5 includes stiffening tabs or strips provided along two opposite sides of the patch. In one embodiment, the strips are applied to the anterior and posterior portions of the patch. The strips are sized and positioned so as to still allow for the medial row of sutures, as described above.

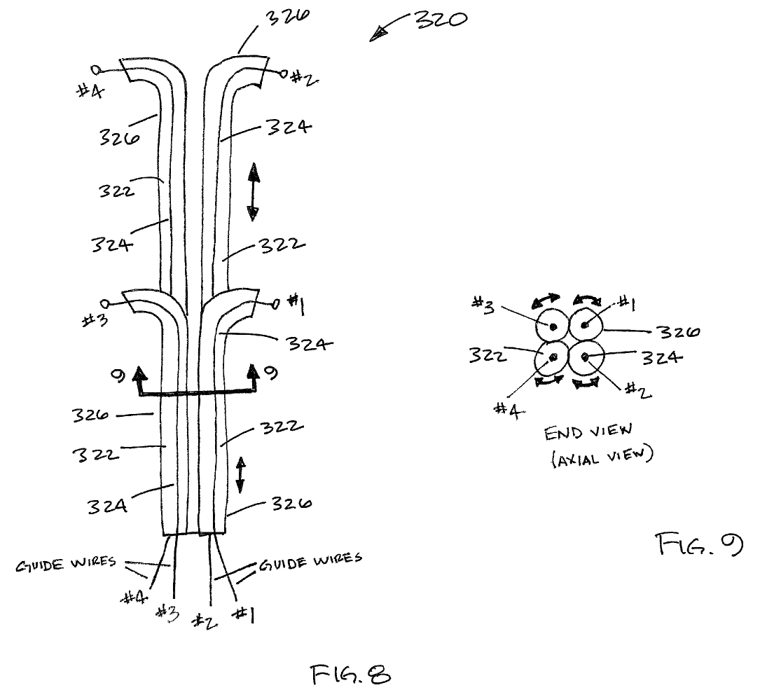

In one embodiment of use, the patch (with the strips) is rolled up longitudinally and fed into the cannula to facilitate delivery into the subacromial space of the joint. Once in the subacromial space, the strips provide medial to lateral rigidity, thereby giving a natural sense of shape to the patch while still allowing free floating. In one embodiment, the medial sutures are tied, spinal needles are inserted, and a lateral row of suture anchors are used to anchor the sutures in a standard fashion (FIG. 6). In one embodiment, the stiffening tabs or strips are formed of a bioabsorbable material with a short half-life so as to degrade over time. An example of such a material includes corn starch.

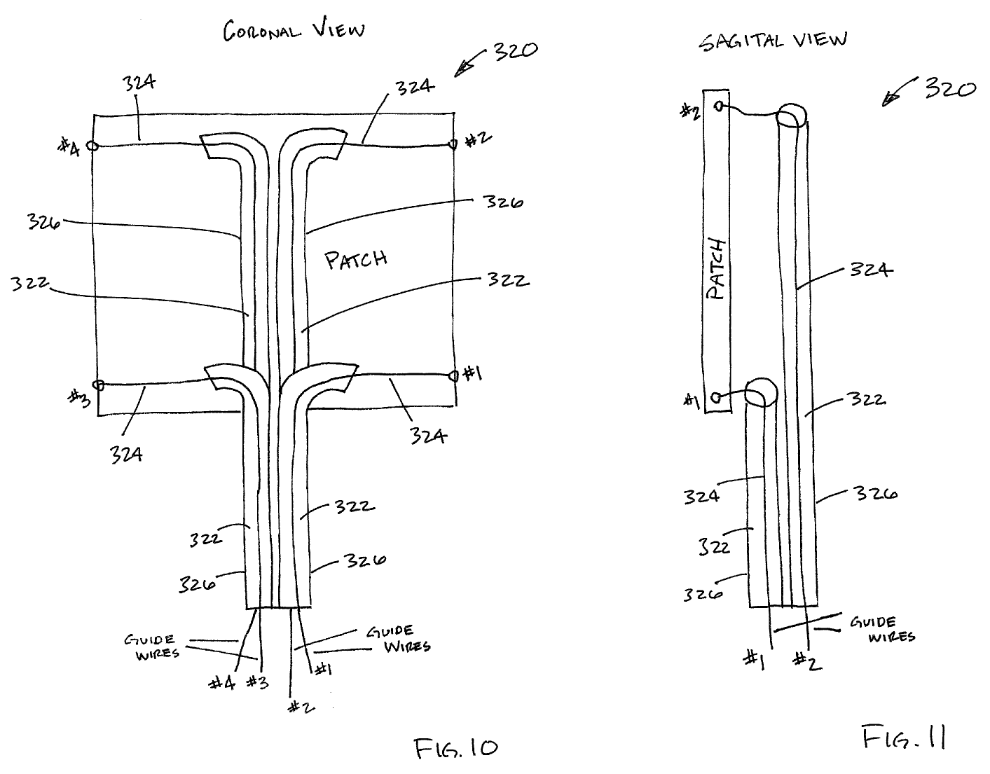

In another embodiment, as illustrated in FIG. 7, an additional stiffening tab or strip is provided along a third side of the patch. This construct provides additional rigidity to help deploy and “unroll” the patch such that the patch returns to its original rectangular shape after delivery into the subacromial space of the joint.

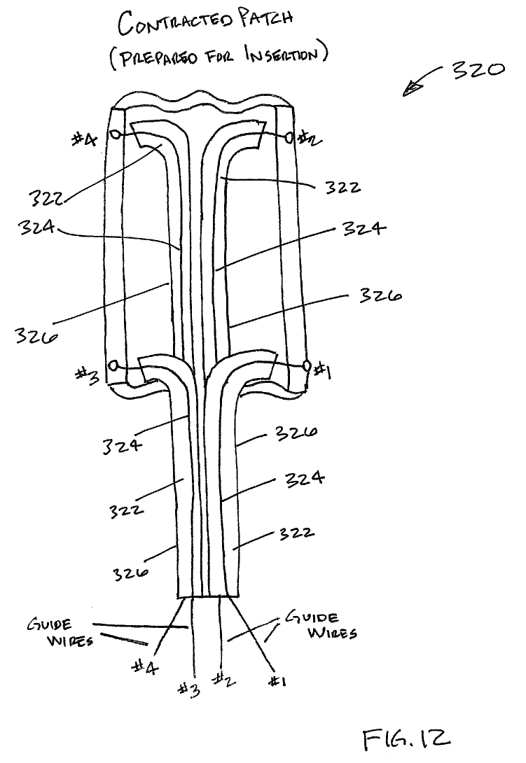

FIGS. 8 and 9 illustrate another embodiment of a rotator cuff patch delivery device according to the present invention. The rotator cuff patch delivery device of FIGS. 8 and 9 includes a plurality of channels each configured to slidably receive a guide wire. In one embodiment, the rotator cuff patch delivery device includes an arrangement of four channels each configured to receive a separate guide wire.

In one embodiment, the channels of the rotator cuff patch delivery device are formed by generally L-shaped tubes or guides. In one embodiment, the arrangement of four channels is formed by two longer tubes and two shorter tubes collectively grouped and/or attached to each other. In one embodiment, the two longer tubes and the two shorter tubes are of lengths corresponding to a size of the patch so as to generally coincide with the four corners of the patch.

In one embodiment, the tubes are grouped together such that a relative position and, therefore, a relative length of the tubes along a longitudinal axis is adjustable. For example, the relative length of the longer tubes and/or the shorter tubes may be adjusted to accommodate different size patches and/or facilitate insertion and deployment of the patch, as described below. In addition, in one embodiment, the tubes are rotatable relative to each other about a respective longitudinal axis to facilitate insertion and deployment of the patch.

In one embodiment, each of the guide wires include a preloaded wire such as a nitinol wire. In one embodiment, a loop is provided at the end of each nitinol wire to facilitate attachment of the patch to the rotator cuff patch delivery device, as described below.

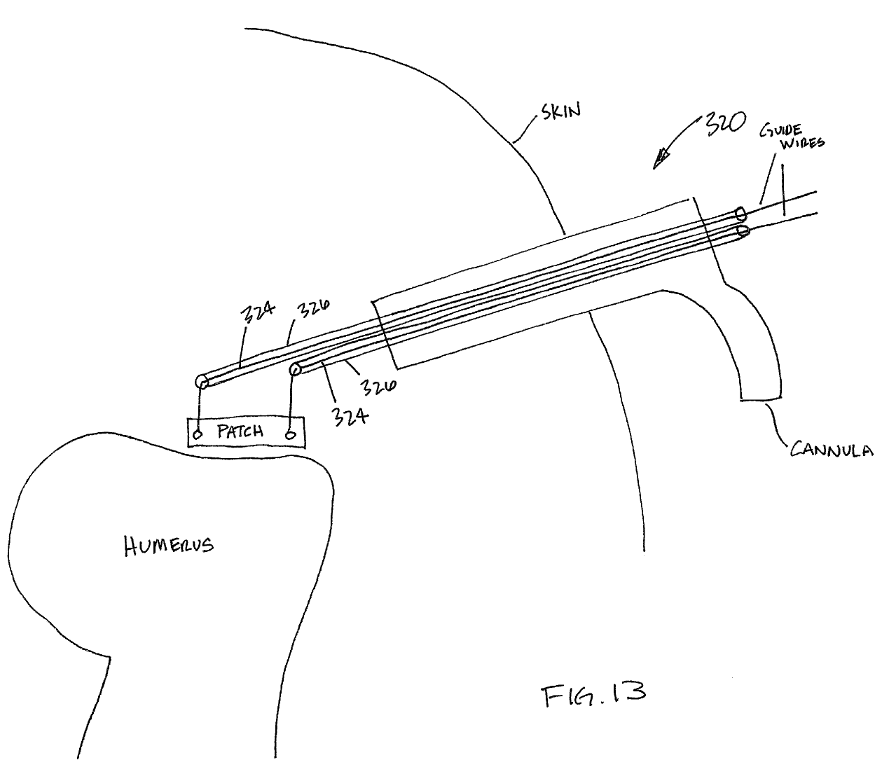

In one embodiment of use, as illustrated in FIGS. 10 and 11, an end of each guide wire is secured to a respective corner of the allograft patch. In one embodiment, the ends of the guide wires are secured to the patch by provisional sutures.

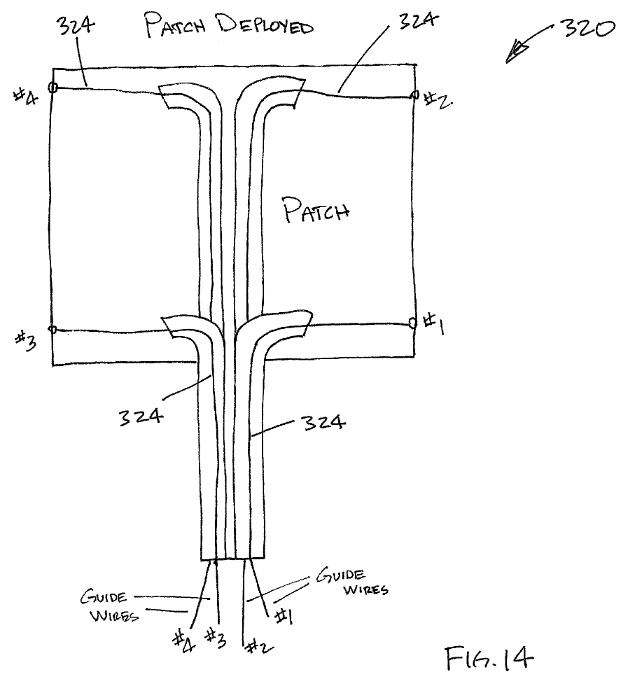

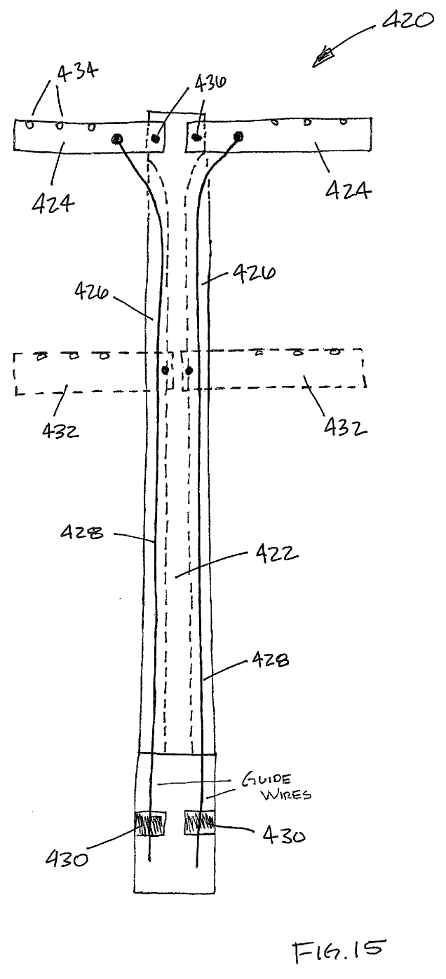

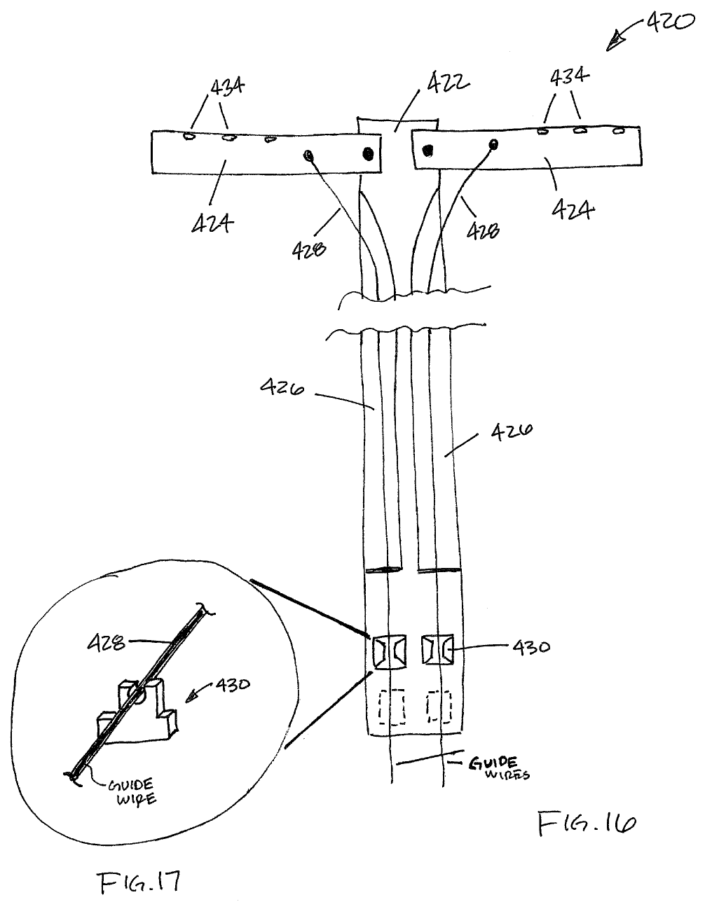

In one embodiment, as illustrated in FIG. 12, after the guide wires are attached to the patch, the guide wires are drawn through the respective tubes and away from the patch so as to draw the corners of the patch inward toward the tubes. As such, as illustrated in FIG. 13, the construct including the tubes and the patch may be inserted through a cannula and into the subacromial space of the shoulder. Once in the subacromial space of the shoulder, the guide wires are advanced into the respective tubes thereby deploying and spreading out the patch to its approximate size and shape (FIG. 14). After the patch is secured, as described above, the guide wires may be detached (i.e., cut away) from the patch and removed from the shoulder space.

Although illustrated and described as including four channels formed by four tubes, it is within the scope of the present invention for the rotator cuff patch delivery device to include any number of channels formed by any number of tubes or guides.

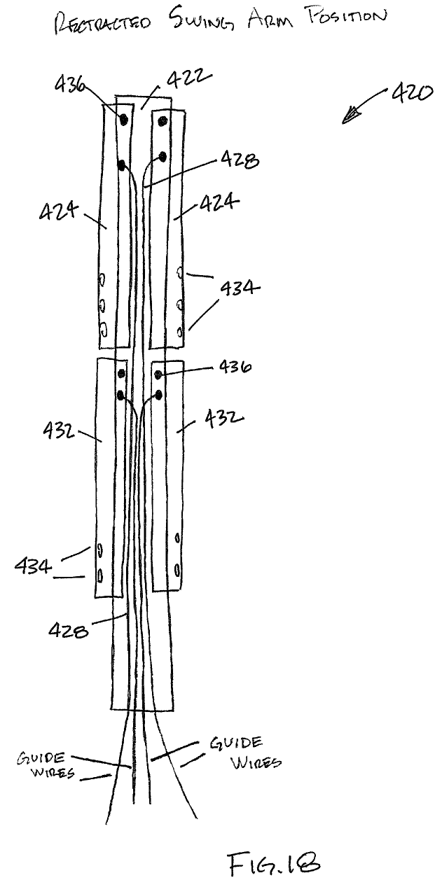

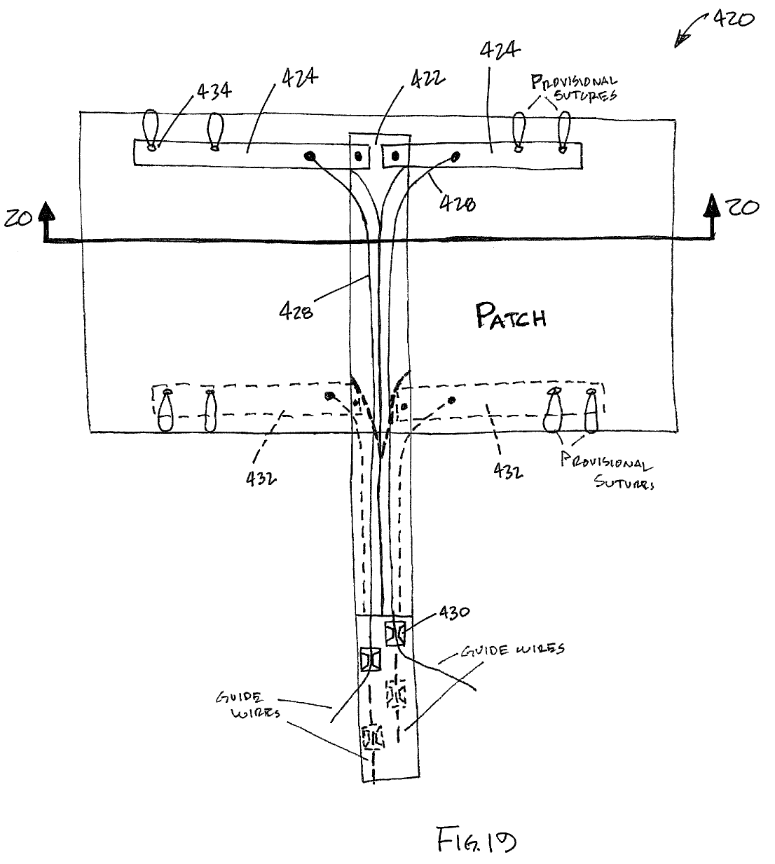

FIGS. 15, 16, 17, and 18 illustrate another embodiment of a rotator cuff patch delivery device according to the present invention. The rotator cuff patch delivery device of FIGS. 15, 16, 17, and 18 includes a central post with two swing arms attached at the end of the post. In one embodiment, the swing arms are pivotally attached to one end of the post such that the post and the swing arms form a generally “T-shaped” arrangement when the swing arms are deployed.

In one embodiment, the post of the rotator cuff patch delivery device includes one or more channels or guides formed through or by the central post, and the swing arms are retracted and deployed by guide wires attached to the swing arms and running through the channels. In one embodiment, the guide wires may be inserted into a set of cleats or locking features (FIG. 17) in order to keep the swing arms in the desired position. The locking features may be formed, for example, of a material such as rubber, plastic, or silicone which holds the guide wires in the desired position. In one embodiment, each of the guide wires include a preloaded wire such as a nitinol wire.

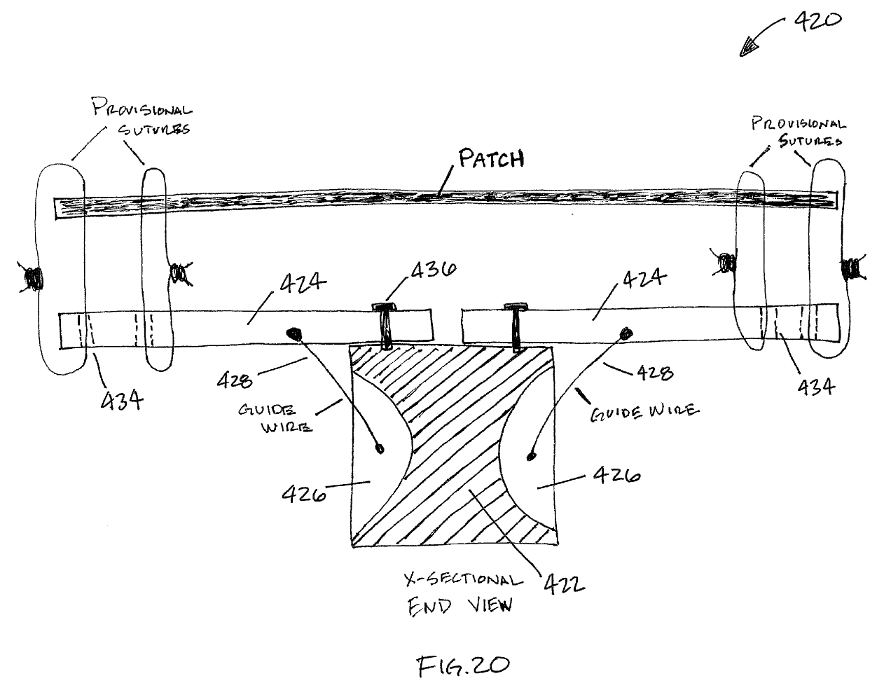

In one embodiment of use, as illustrated in FIGS. 19 and 20, prior to placing the patch in the subacromial space of the joint, the patch is secured to the swing arms of the rotator cuff patch delivery device with the swing arms in the deployed “T” position. In one embodiment, the patch is secured to the swing arms with provisional sutures using suture throughholes provided in the swing arms.

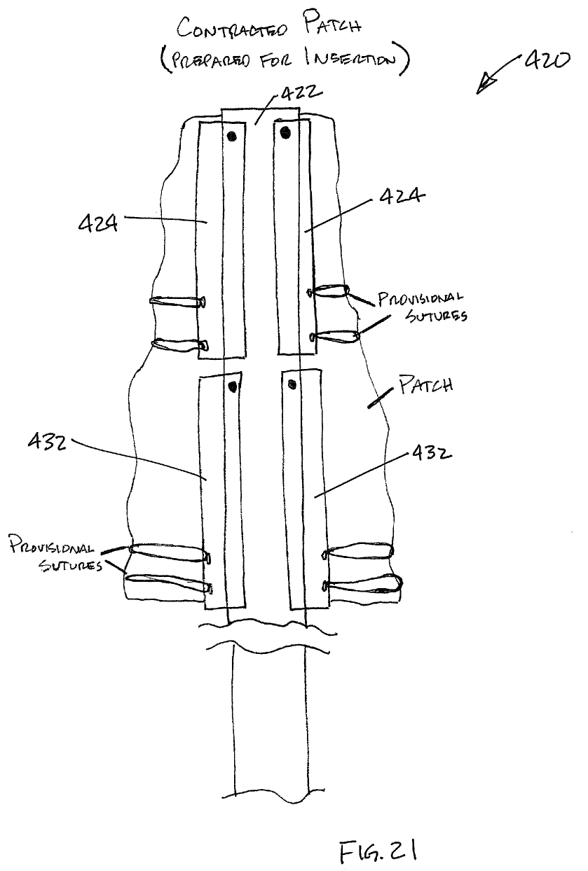

In one embodiment, as illustrated in FIG. 21, with the patch secured to the swing arms, the swing arms are then retracted using the guide wires by pulling on the guide wires and drawing the guide wires through the respective channels and away from the patch. As such, the patch is retracted inward toward the post.

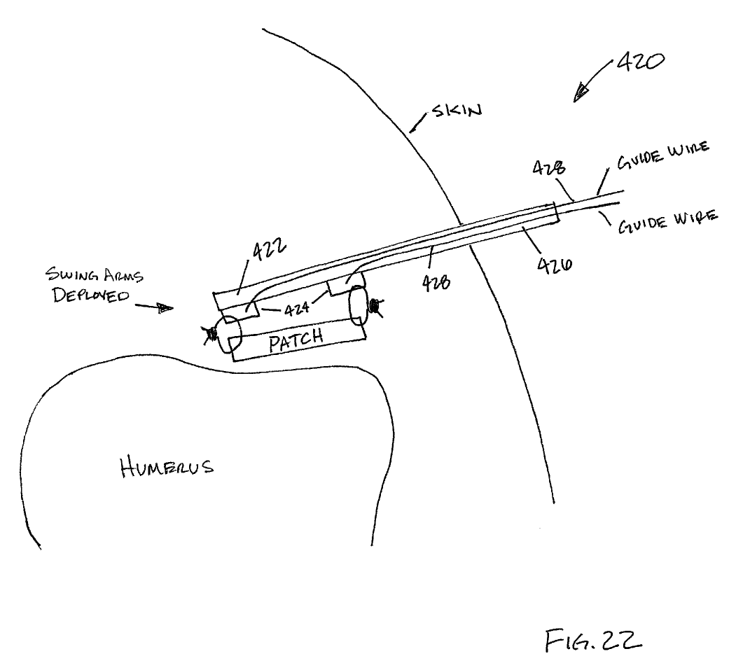

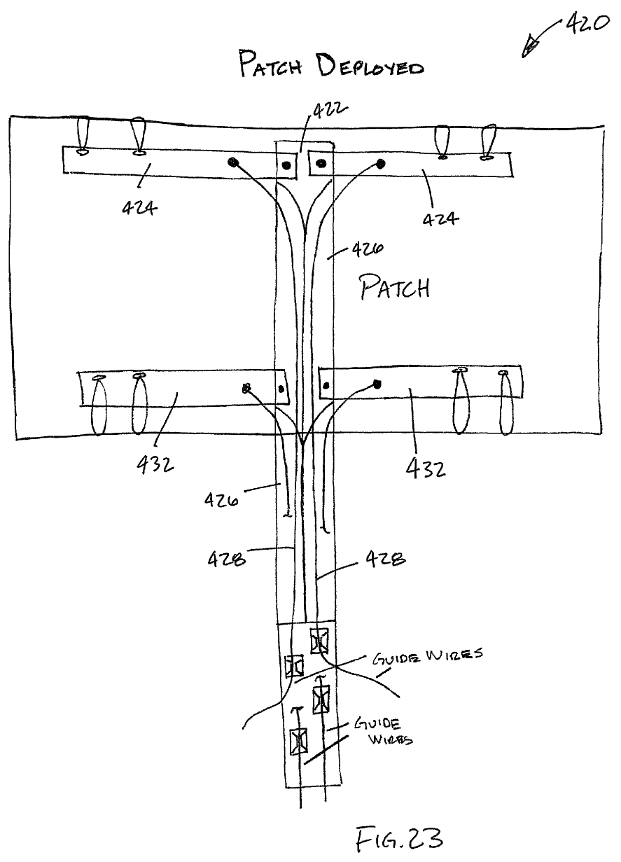

In one embodiment, as illustrated in FIG. 22, once the patch and the rotator cuff patch delivery device are at their most compact state, the whole construct is introduced into the subacromial space. Thereafter, the guide wires are advanced into the respective channels in order to re-deploy the patch (FIG. 23). Final placement and fixation of the patch may be performed, as described above.

In one embodiment, a second set of swing arms are pivotally attached to the post of the rotator cuff patch delivery device more laterally, away from the end of the central post. The second set of swing arms provide for additional attachment of the patch to the rotator cuff patch delivery device, and are operated in a manner similar to that described above. In one embodiment, to accommodate the additional swing arms, two additional channels are provided through the central post of the rotator cuff patch delivery device to house additional guide wires for deployment of the additional swing arms.

Although illustrated and described as including two or four swing arms with a corresponding two or four channels, it is within the scope of the present invention for the rotator cuff patch delivery device to include any number of swing arms and any number of channels or guides.

Embodiments of a rotator cuff patch delivery device illustrated and described herein serve to assist an orthopaedic surgeon with the delivery of an allograft patch into the subacromial space without having the allograft patch become twisted or entangled. The device would be easy to apply and inexpensive to use. It would improve visualization and cut down on operative time.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.
Reference numerals in the Figures are identified as follows:
120—rotator cuff patch delivery device
122—pad portion
124—inflatable chamber portion
126—fluid influx tube
220—rotator cuff patch delivery device
222—stiffening strips
224—stiffening strip
320—rotator cuff patch delivery device
322—channels
324—guide wires
326—tubes
420—rotator cuff patch delivery device
422—central post
424—swing arms
426—channels
428—guide wires
430—locking features
432—swing arms
434—suture throughholes
436—pivots

What is claimed is:

1. A device for delivering an allograft patch through a cannula, comprising:
a plurality of channels; and
a plurality of guide wires slidably received within the channels.

2. The device of claim 1, wherein the guide wires are configured to be drawn through the channels away from the allograft patch to retract the allograft patch toward the channels for feeding of the device and the allograft patch through the cannula.

3. The device of claim 1, wherein the guide wires are configured to be advanced into the channels toward the allograft patch to deploy the allograft patch.

4. The device of claim 1, wherein the plurality of channels includes two channels each configured to receive at least one of the guide wires.

5. The device of claim 1, wherein the plurality of channels includes four channels each configured to receive a separate one of the guide wires.

6. The device of claim 1, wherein each of the guide wires includes a preloaded wire.

7. The device of claim 1, wherein an end of each of the guide wires is configured to be secured to the allograft patch.

8. The device of claim 1, wherein the plurality of channels are formed by a plurality of L-shaped tubes.

9. The device of claim 8, wherein the L-shaped tubes are collectively grouped and attached to each other.

10. The device of claim 8, wherein a length of the L-shaped tubes relative to each other is adjustable along a longitudinal axis.

11. The device of claim 8, wherein the L-shaped tubes are rotatable relative to each other about a respective longitudinal axis.

12. The device of claim 8, wherein the plurality of L-shaped tubes includes an arrangement of two longer tubes and two shorter tubes.

13. The device of claim 1, wherein the plurality of channels are formed in a central post, and wherein the device further comprises at least two swing arms pivotally attached to the central post, wherein the allograft patch is configured to be secured to the swing arms.

14. The device of claim 13, wherein the guide wires are attached to the swing arms, and wherein the swing arms are configured to be retracted and deployed with the guide wires.

15. The device of claim 13, wherein the swing arms are configured to extend from and form a T-shaped arrangement with the central post when deployed, and configured to be drawn toward the central post when retracted.

16. The device of claim 13, wherein the at least two swing arms comprises a first set of swing arms pivotally attached to the central post adjacent one end thereof, and a second set of swing arms pivotally attached to the central post away from the one end thereof.

17. The device of claim 1, wherein the device further comprises a locking feature configured to hold an associated one of the guide wires in a desired position.

18. A device for delivering an allograft patch through a cannula, comprising:
means for guiding a plurality of guide wires configured to be drawn away from the allograft patch to retract the allograft patch for feeding of the allograft patch through the cannula and configured to be advanced toward the allograft patch to deploy the allograft patch.

19. The device of claim 18, wherein the means comprises a plurality of L-shaped tubes collectively grouped and attached to each other, wherein the guide wires are slidably received within the L-shaped tubes.

20. The device of claim 18, wherein the means comprises a central post and at least two swing arms pivotally attached to the central post, wherein the guide wires are attached to the swing arms and slidably received within the central post.
